(19) United States
(12) Patent Application Publication
(10) Pub. No.: US 2016/0067066 A1
(43) Pub. Date: Mar. 10, 2016

(54) ANTITHROMBOTIC ARTIFICIAL BLOOD VESSEL

(71) Applicant: TORAY INDUSTRIES, INC., Tokyo (JP)

(72) Inventors: Kazuhiro Tanahashi, Otsu (JP); Yuka Sakaguchi, Otsu (JP); Masaki Fujita, Otsu (JP); Koji Kadowaki, Otsu (JP); Hiroshi Tsuchikura, Otsu (JP); Satoshi Yamada, Otsu (JP)

(21) Appl. No.: 14/783,956

(22) PCT Filed: Apr. 10, 2014
(86) PCT No.: PCT/JP2014/060376

§ 371 Date: Oct. 12, 2015

(30) Foreign Application Priority Data
Apr. 12, 2013 (JP) 2013-084121

(51) Int. Cl. A61F 2/86

(52) U.S. Cl. CPC A61F 2/86; A61F 2210/0076; A61F 2250/0067

(57) ABSTRACT

An artificial blood vessel promotes intimal formation after indwelling, and is capable of maintaining antithrombogenicity during the intimal formation and maintaining its patency for a long time. The artificial blood vessel is a tubular fabric including a fiber layer containing an ultrafine fiber(s) and an ultrafine fiber layer in the inside of the fiber layer, the ultrafine fiber layer being composed of an ultrafine fiber(s) having a fiber diameter(s) of 10 nm to 3 μm, wherein an antithrombin agent having a polymer chain other than heparin is covalently bound to the ultrafine fiber via the polymer chain, and the thrombin activity inhibition rate on the fiber surface at 37° C. is not less than 60%.

### TECHNICAL FIELD

This disclosure relates to an artificial blood vessel to be used for reconstruction, repair, or replacement of a blood vessel that has undergone damage and/or the like.

### BACKGROUND

The number of patients suffering from arteriosclerosis is increasing due to population aging and an increase in the population with metabolic syndrome. Arteriosclerosis is an abnormality of arterial walls. In arteriosclerosis, a hyperglycemic state or hyperlipidemic state of blood causes degeneration of the vascular wall and, as a result, the vascular wall becomes weak or thickened, or calcification occurs to make the vascular wall hard and fragile. Although such blood vessel degeneration may occur at any site in the blood vessels in the body, peripheral blood vessels are especially remarkably affected by the degeneration.

Treatment of such a degenerated blood vessel is conventionally carried out by a minimally invasive endovascular treatment such as balloon dilation or stent placement using a catheter, or by surgery for replacement of the damaged blood vessel with a blood vessel of the patient him or herself or with an artificial blood vessel.

However, when an artificial blood vessel is used, the body recognizes the artificial blood vessel as a foreign substance, and blood clotting reaction proceeds on the blood-contacting surface of the artificial blood vessel to form a thrombus.

A blood vessel in the body has an intima having vascular endothelial cells on its surface contacting with blood, and the intima plays a role in inhibiting formation of a thrombus. Also, in an indwelling artificial blood vessel, vascular endothelial cells cover the blood-contacting surface of the artificial blood vessel to form an intima. However, since the artificial blood vessel is recognized as a foreign substance until the intima is covered with the endothelial cells, means for preventing thrombus formation is required until formation of the intima. In particular, at a site where an artificial blood vessel having a small diameter is used, the blood flow is low so that deposition of thrombi easily occurs, and the blood vessel is likely to be clogged even with a small amount of thrombi. At present, the long-term performance of artificial blood vessels having small diameters is not good, and none of such artificial blood vessels is applicable for clinical use.

To solve these problems, development of artificial blood vessels has been conventionally carried out focusing on early intimal formation and early establishment of antithrombogenicity.

Examples of methods of promoting intimal formation include a method in which a growth factor or an inducer of cells is carried by the artificial blood vessel, and a method in which an artificial blood vessel containing, as its constitutional material, a fabric, knit, or non-woven fabric of a fiber such as a polyester fiber is used. In particular, it is known that, when an ultrafine fiber of less than 10 μm is included, the size of the ultrafine fiber or the size of the fiber gap is suitable for cell growth or cell infiltration (JP 1875991 B, JP 1870688 B, and JP 1338011 B). It is also known that ultrafine fibers have effects to promote adhesion of platelets and prevent leakage of blood from the blood vessel wall when the fibers are indwelling (JP 4627978 B).

In a conventional method of imparting antithrombogenicity to an artificial blood vessel, heparin is carried by the artificial blood vessel. Since the fiber itself does not have a capacity to carry heparin, the artificial blood vessel is made to carry a sufficient amount of heparin by a known method such as a method in which a gel composed of a biodegradable polymer or gelatin containing heparin is filled into fibers (JP 3799626 B), or a method in which heparin is immobilized on the fiber surface by covalent bonds (Japanese Translated PCT Patent Application Laid-open No. 2009-545333).

On the other hand, examples of known methods of imparting antithrombogenicity using a substance other than heparin include methods in which an antithrombin agent or a polymer containing an antithrombin agent is bound to the surface using a high-energy ray such as γ-ray (WO 08/032758 and WO 2011/078208).

However, when fiber gaps are filled such as in the artificial blood vessel described in JP 3799626 B, cellular infiltration is prevented to cause a delay in intimal formation and, furthermore, platelets adhere to gelatin and the like to rather promote thrombus formation, which is problematic. When heparin is immobilized on the fiber surface by covalent bonds such as in the artificial blood vessel described in Japanese Translated PCT Patent Application Laid-open No. 2009-545333, the amount of heparin that can be bound to the surface is limited because of the large molecular weight of heparin, and there is no long-term effect, which is problematic. Moreover, there is a problem that the cellular adhesiveness decreases since heparin, which has very high hydrophilicity, is bound to the fiber surface.

There are also methods as described in WO 08/032758 and WO 2011/078208, in which an antithrombin agent is bound to the surface by a high-energy ray such as γ-ray, but such methods have a problem that the antithrombin agent is degraded to cause a decrease in the activity, resulting in an insufficient antithrombotic performance. The reaction for immobilization on the surface of the base material is achieved by γ-ray irradiation in a state where the antithrombin agent is adsorbed on the surface in an aqueous solution. However, an aqueous solution with high surface tension does not permeate into gaps of polyester ultrafine fibers having high hydrophobicity, and this results in unevenness of the surface treatment of the fiber surface in the inner portion, leading to induction of thrombogenic response in the portion that has not undergone the surface treatment. Moreover, since a hydrophilic polymer does not adhere to polyester, adherence of the antithrombin agent does not occur unless the antithrombin agent itself has affinity to polyester so that the amount of the agent immobilized on the surface may decrease. Moreover, since the antithrombin agent is bound to the surface without intermediation by a hydrophilic polymer as a spacer, there is only a low degree of spatial freedom, and binding with thrombin is therefor limited, which is problematic.

Thus, conventional artificial blood vessels have failed in simultaneous achievement of cellular affinity and antithrombogenicity and, in particular, there is no artificial blood vessel having a small diameter that is available for long-term clinical use at present in the world.

It could therefore be helpful to provide an artificial blood vessel which promotes intimal formation after indwelling, and is capable of maintaining antithrombogenicity during intimal formation and maintaining its patency for a long time.

### SUMMARY

We discovered that, by covalent bonding of an antithrombin agent having a polymer chain other than heparin to an ultrafine fiber via the polymer moiety, antithrombogenicity can be imparted while the fine structure composed of the ultrafine fiber is maintained, that is, both cellular affinity and antithrombogenicity can be realized.

We thus provide the following (1) to (12).

(1) An artificial blood vessel which is a tubular fabric comprising a fiber layer containing an ultrafine fiber(s) and an ultrafine fiber layer in the inside of the fiber layer, the ultrafine fiber layer being composed of an ultrafine fiber(s) having a fiber diameter(s) of not less than 10 nm and not more than 3 μm, wherein an antithrombin agent having a polymer chain other than heparin is covalently bound to the ultrafine fiber via the polymer chain, and the thrombin activity inhibition rate on the fiber surface at 37° C. is not less than 60%.

(2) The artificial blood vessel according to (1), wherein the molecular weight of the antithrombin agent is not more than 3000.

(3) The artificial blood vessel according to (1) or (2), whose water permeability at 120 mmHg is not less than 100 mL/cm²/min and less than 4000 mL/cm²/min.

(4) The artificial blood vessel according to any one of (1) to (3), wherein the thrombin activity inhibition rate in an extract obtained by 24 hours of extraction at 37° C. in 10 mL of physiological saline per 1 g of the artificial blood vessel is less than 5%.

(5) The artificial blood vessel according to any one of (1) to (4), wherein the antithrombin agent has a guanidino group, guanido group, and/or amidino group.

(6) The artificial blood vessel according to any one of (1) to (5), wherein the polymer chain is a polymer structure selected from polyalkylene glycol, polyvinyl alcohol, and polyvinyl pyrrolidone.

(7) The artificial blood vessel according to any one of (1) to (6), wherein the antithrombin agent is selected from Chemical Formulae (I) to (IV):

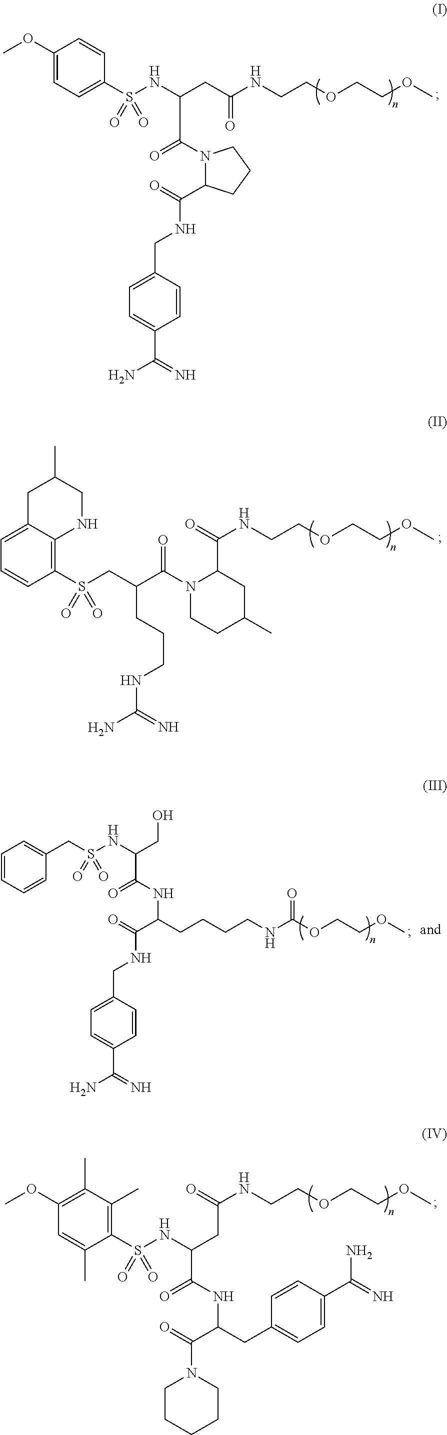

(wherein n represents an integer of 1 to 500).

(8) The artificial blood vessel according to any one of (1) to (7), wherein the fiber layer is composed of the ultrafine fiber(s) and a multifilament(s) having a total fineness of 1 to 60 decitex.

(9) The artificial blood vessel according to (8), wherein the fineness of single yarns constituting the multifilament is 0.5 to 10.0 decitex.

(10) The artificial blood vessel according to any one of (1) to (9), having a platelet adhesion rate of less than 20%.

(11) The artificial blood vessel according to any one of (1) to (10), wherein the tubular fabric is composed of a polyester fiber(s).

(12) The artificial blood vessel according to any one of (1) to (11), wherein the inner diameter of the tubular fabric is not less than 1 mm and less than 10 mm.

An artificial blood vessel which promotes intimal formation after indwelling, and is capable of maintaining antithrombogenicity during intimal formation and maintaining its patency for a long time can be provided.

### BRIEF DESCRIPTION OF THE DRAWING

FIG. 1 is a schematic diagram showing the fiber structure of the artificial blood vessel.

### DESCRIPTION OF SYMBOLS

1 . . . Ultrafine fiber, 2 . . . Fiber layer, 3 . . . Ultrafine fiber layer

### DETAILED DESCRIPTION

The ultrafine fiber means a fiber having a fiber diameter of not less than 10 nm and not more than 3 μm. When an artificial blood vessel having an ultrafine fiber is used, the number of scaffolds suitable for adhesion of living cells remarkably increases because of the extreme fineness of the fiber and excellent cellular infiltration can be achieved. Favorable intimal formation occurs in an extremely early phase and leakage of blood hardly occurs.

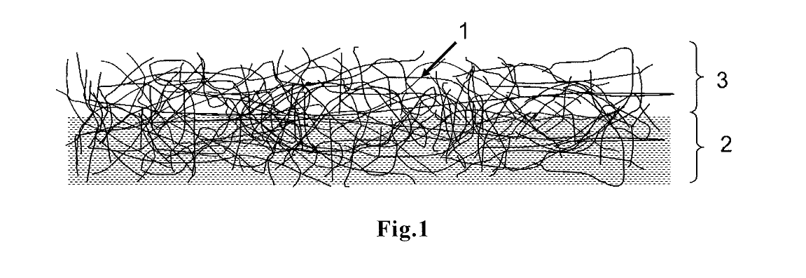

Since the strength that allows the artificial blood vessel to follow blood pressure and movement of tissues cannot be exerted with only ultrafine fibers, the artificial blood vessel has a fiber structure composed of, as shown in FIG. 1, a fiber layer 2 in which ultrafine fibers 1 are dispersed in gaps of a basic tissue formed by a coarse texture, stitch or the like constituted of thick fibers; and an ultrafine fiber layer 3 composed of ultrafine fibers 1 inside the fiber layer 2. The artificial blood vessel is formed by making this fiber structure into a tubular shape.

As a production method of forming the fiber structure in which ultrafine fibers are dispersed in gaps of a basic tissue formed by a coarse texture, stitch or the like, a common production method for ultrafine fibers may be employed. Together with a fiber having a size suitable for the strength of the basic tissue, a multicomponent fiber having a sea-island structure is subjected to weaving, knitting, or processing into a braid or a non-woven fabric, and then the sea structure of part of the multicomponent fiber is dissolved using an alkali or the like to perform ultrafining treatment. By this, the ultrafine fiber in the base fabric and the ultrafine fiber layer are preferably prepared.

Thereafter, a gap structure which is more desirable for cells can be achieved by interlacing the ultrafine fiber with the basic tissue by a water-jet process, air-jet process or the like. To allow more effective exertion of the cellular affinity, formation of the ultrafine fiber layer on the blood-contacting surface can be further promoted by a method in which, for example, the blood-contacting surface is rubbed with a file to fuzz the surface.

The fiber material is not limited as long as it is a polymer having biocompatibility. Examples of the fiber material include polyester, polyethylene, polytetrafluoroethylene, polyurethane, polyamide, and nylon. Among these fiber materials, polyester, especially polyethylene terephthalate, is preferred since it has been conventionally clinically used as a material of artificial blood vessels and has excellent strength.

The fiber may be in any form and examples of the form include a spun yarn, multifilament yarn, monofilament yarn, and film split fiber yarn. From the viewpoint of strength, uniformity of physical properties and flexibility, a multifilament yarn is excellent as the form of the fiber. The yarn may be either untwisted or twisted. The yarn may be crimped to a certain extent.

The total fineness of the fiber is preferably 1 to 60 decitex (Dtex), more preferably 1 to 40 decitex. The lower limit of the total fineness is more preferably 5 decitex, most preferably 10 decitex. The upper limit of the total fineness is more preferably 35 decitex, most preferably 25 decitex. With a total fineness of not less than 1 decitex, the strength required for the base fabric can be maintained and, with a total fineness of not more than 40 decitex, the thickness of the base fabric can be reduced.

The single yarn fineness is preferably 0.5 to 10 decitex (Dtex), more preferably 0.5 to 3.0 decitex. The lower limit of the single yarn fineness is more preferably 1 decitex, and the upper limit of the single yarn fineness is more preferably 2 decitex. When the single yarn fineness is not less than 3 decitex, flexibility is deteriorated. When the single yarn fineness is not more than 0.5 decitex, the hydrolysis rate is high and there is a problem of deterioration of the strength.

In the tubular fabric which forms the artificial blood vessel, the cloth is provided as a fabric because of its excellent dimensional stability and strength.

To increase the amount of the antithrombin agent immobilized on the fiber surface, ultrafine single-yarn-fineness multifilament yarns may be effectively placed in a part of the cloth. The single-yarn fiber diameter of this ultrafine single-yarn-fineness multifilament yarns is preferably 10 nm to 20 μm, more preferably 10 nm to 3 μm, most preferably 0.8 to 1.2 μm.

The size and the amount of the fiber gaps in the fiber layer and the ultrafine fiber layer of the artificial blood vessel can be represented using as an index the water permeability under a pressure of 120 mmHg, and the fiber gap is preferably 100 mL/cm²/min. to 4000 mL/cm²/min. To form an intima containing a stable vascular endothelial cell layer on the blood-contacting surface of the artificial blood vessel, a cell layer which supports the intima and mainly contains vascular smooth muscle and fibroblasts is important. Cells in this cell layer, together with vascular endothelial cells that migrate on the surface of the blood vessel, pass through fiber gaps, and infiltrate from the anastomotic site into the inside. Vascular endothelial cells not only infiltrate from the anastomotic site, but also infiltrate from sites on the inner wall of the artificial blood vessel where openings are formed by blood capillaries that infiltrated from the outer wall of the artificial blood vessel through fiber gaps.

In view of this, the fiber gap is preferably not less than 100 mL/cm²/min since, in such cases, intimal formation due to infiltration of the inside of the fiber layer by cells and blood capillaries easily occurs. When the fiber gap is not more than 4000 mL/cm²/min., cellular pseudopodia more easily reach the inside of the fiber layer, and fill the gaps to prevent blood leakage, which is preferred.

The size of the artificial blood vessel is not limited. The artificial blood vessel is most effective as a thin artificial blood vessel having an inner diameter of not less than 1 mm and less than 10 mm.

Our artificial blood vessel realizes both cellular affinity and antithrombogenicity since an antithrombin agent having a polymer chain other than heparin is covalently bound via the polymer chain to the surface of the fiber and the ultrafine fiber constituting the basic tissue.

Among antithrombin agents having a polymer chain other than heparin, a low-molecular-weight antithrombin agent is preferably used. More specifically, an antithrombin agent having a molecular weight of not more than 3000 is preferred.

Since heparin, which is widely known as an antithrombin agent, is a large molecule having a molecular weight of 30,000 to 35,000 daltons, it can be immobilized on the surface in only a limited amount. Although low-molecular-weight heparins whose molecular weights are lower than that of heparin are also clinically used, even these low-molecular-weight heparins have molecular weights as large as 4,000 to 6,000, which are about 10 times the molecular weights of synthetic antithrombin substances. Heparin can inhibit the activity of thrombin only after binding to antithrombin III and thrombin. Since the binding sites of antithrombin III and thrombin are separately present in the molecule, it is very difficult to control immobilization on the surface while allowing these binding sites to be arranged in optimum positions. This difficulty also causes the low reaction efficiency of immobilization on the surface. Moreover, the antithrombin activity itself of heparin is about 10 times lower than those of synthetic antithrombin substances. Thus, the activity of heparin is originally low. Moreover, it is known that there are, in the world, not a small number of patients with heparin-induced thrombocytopenia, who show excessive allergy to heparin. Heparin cannot be used completely freely when there is a social or ethical reason.

The antithrombin activity substance having a polymer chain is preferably a substance having a guanidino group, guanido group, and/or amidino group, more preferably a substance selected from General Formulae (I) to (IV):

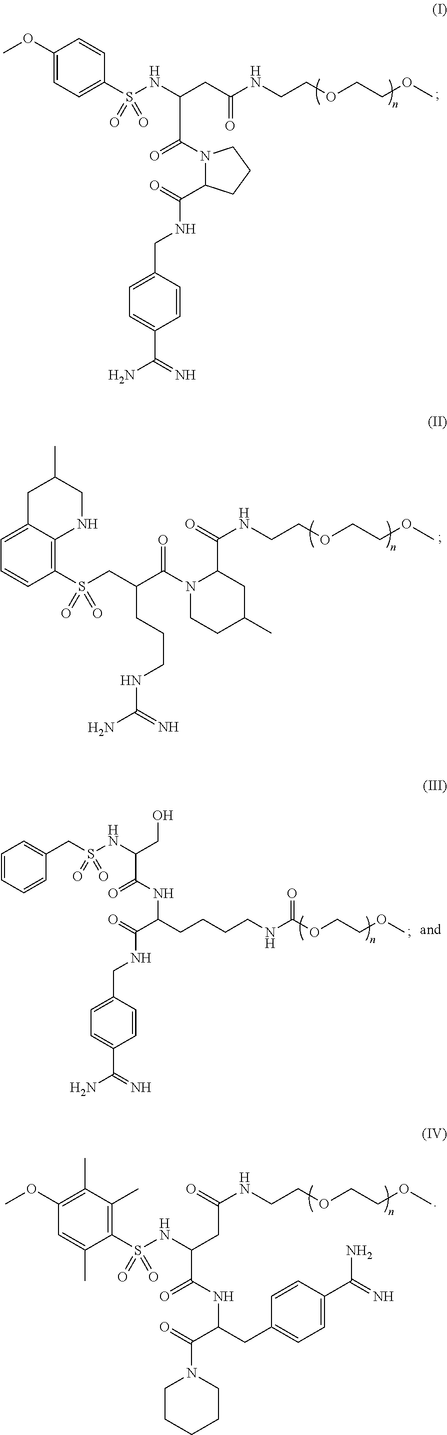

Since these antithrombin agents having a polymer chain are immobilized on the fiber surface by covalent bonding via a functional group at the end of the polymer chain, elution of the agents does not occur and the agents can maintain their effects on the surface for a long time. The most preferred method of immobilizing the antithrombin agent on the fiber surface by covalent bonding while maintaining its antithrombin activity is a method in which the reactive functional group to be used for the immobilization reaction is introduced to a site distant from the active site of the antithrombin agent, for example, to a site on the opposite side with respect to the polymer chain to provide a derivative, and a chemical reaction such as condensation reaction, addition reaction, or graft polymerization is then performed to achieve immobilization. Methods in which the antithrombin agent and the fiber, in their coexistence, are irradiated with a high-energy ray such as γ-ray or electron beam, and methods in which the fiber is subjected to plasma treatment and then brought into contact with the antithrombin agent, cannot be used since a highly reactive radical species deactivates the active site, or the orientation cannot be controlled such that the antithrombin agent can exert the maximum activity.

Immobilization of the antithrombin agent is preferably carried out by a method in which the distance between the immobilization site and the active site is as long as possible, and the reactive functional group is introduced via the polymer chain to secure freedom after immobilization. The polymer chain preferably has hydrophilicity. The structure of the hydrophilic polymer chain is not limited, and is preferably a high molecular structure selected from polyalkylene glycols such as polyethylene glycol (PEG), polypropylene glycol (PPG), and polyethylene glycol/polypropylene glycol copolymers (PEG-PPG); polyvinyl alcohol (PVA); and polyvinyl pyrrolidone (PVP); for exertion of the effect especially in blood, which is hydrophilic. The degree of polymerization of the hydrophilic polymer chain, n, is not limited, and is preferably 1 to 500 since, when n is too large, the hydrophilicity increases and, therefore, cellular adhesiveness decreases.

Antithrombogenicity and cellular affinity of the artificial blood vessel are shown by measurement of water permeability, thrombin activity inhibition rate in an extract, thrombin activity inhibition rate on the fiber surface, platelet adhesion rate, cell adhesion rate, and thrombus adhesion. These are measured by the following methods.

### Water Permeability

Two sites are randomly sampled from the artificial blood vessel, and measurement is carried out twice for each sample by the method described below, followed by calculating the arithmetic mean of the measured values. The artificial blood vessel was cut along the axial direction, and a sample piece having a size of 1 cm×1 cm was prepared. Between two doughnut-shaped packings with a diameter of 4 cm on each of which a hole having a diameter of 0.5 cm is formed by punching, the fabric sample having a size of 1 cm×1 cm is sandwiched such that liquid flow is allowed only through the punched portion. The resultant is stored in a housing for a circular filtration filter. Water filtered through a reverse osmosis membrane is passed through this circular filtration filter at a temperature of 25° C. for not less than 2 minutes until the sample piece sufficiently contains water. Under the conditions of a temperature of 25° C. and a filtration differential pressure of 120 mmHg, external-pressure dead-end filtration of water filtered through a reverse osmosis membrane is carried out for 30 seconds to measure the amount of the water (mL) that permeates the portion with a diameter of 1 cm. The permeation volume is calculated by rounding the measured value to an integer. By converting the permeation volume (mL) to the value per unit time (min.) per effective area on the sample piece (cm²), the water permeability at a pressure of 120 mmHg is determined.

To investigate the degree of elution of the antithrombin agent from the artificial blood vessel after immobilization of the antithrombin agent, the thrombin activity inhibition rate in an extract may be measured by the following method. In terms of the thrombin activity inhibition rate in this extract, the thrombin activity inhibition rate in the extract at 37° C. is preferably as low as possible. The thrombin activity inhibition rate is preferably less than 5%, more preferably less than 1%.

### Method of Measuring Thrombin Activity Inhibition Rate in Extract

One gram of a ring-shaped sample prepared by cutting the artificial blood vessel into round slices in the transverse direction is cut into 10 small pieces each having a weight of 0.1 g along the longitudinal direction of the original blood vessel, and extraction is carried out with 10 mL of physiological saline per 1 g of the sample at 37° C. for 24 hours. To 10 μL of sample-free physiological saline, or to 10 μL of the sample extract, 0.5 mL of 0.1 U/mL aqueous thrombin (Haematologic Technologies Inc.) solution and 0.5 mL of 200 μM aqueous S2238 (Sekisui Medical Co., Ltd.) solution are added. After leaving the resulting mixture to stand at 37° C. for 45 minutes, the absorbance at 405 nm is measured using a microplate reader (Corona Electric Co., Ltd., MTP-300). Using the molar extinction coefficient of p-nitroaniline at a wavelength of 316 nm (1.27×10⁴ mol⁻¹·L·cm⁻¹), the amount of S2238 degraded per unit time, that is, the degradation rate of S2238, was calculated. As shown in Equation 1, the ratio of the degradation rate in the extract to the degradation rate in the sample-free physiological saline, which is taken as 100, is determined, to calculate the thrombin activity inhibition rate at 37° C. Equation 1 is used to provide the thrombin activity inhibition rate in the extract.

Thrombin activity inhibition rate(%)=(1−degradation rate in extract/degradation rate in physiological saline)×100 (1)

In the artificial blood vessel after immobilization of the antithrombin agent, the antithrombin performance on the fiber surface can be measured by the following method of measuring the thrombin activity inhibition rate on the fiber surface. In terms of the antithrombin activity inhibition rate on the fiber surface, the thrombin activity inhibition rate is preferably as high as possible. The thrombin activity inhibition rate is preferably not less than 60%, more preferably not less than 80%.

### Method of Measuring Thrombin Activity Inhibition Rate on Fiber Surface

One gram of a ring-shaped sample prepared by cutting the artificial blood vessel into round slices in the transverse direction is cut into 10 small pieces each having a weight of 0.1 g along the longitudinal direction of the original blood vessel, and 5 mL of 0.1 U/mL aqueous thrombin (Haematologic Technologies Inc.) solution and 0.5 mL of 200 μM aqueous S2238 (Sekisui Medical Co., Ltd.) solution per 1 g of the sample are added thereto. After leaving the resulting mixture to stand at 37° C. for 45 minutes, the absorbance at 405 nm is measured using a microplate reader (Corona Electric Co., Ltd., MTP-300). Using the molar extinction coefficient of p-nitroaniline at a wavelength of 316 nm (1.27×10⁴ mol⁻¹·L·cm⁻¹), the amount of S2238 degraded per unit time, that is, the degradation rate of S2238, was calculated. Based on this degradation rate, as shown in Equation 2, the ratio of the degradation rate on the fiber surface to the degradation rate in the sample-free physiological saline, which is taken as 100, is determined, to calculate the thrombin activity inhibition rate at 37° C. Equation 2 is used to provide the thrombin activity inhibition rate on the fiber surface.

Thrombin activity inhibition rate(%)=(1−degradation rate on fiber surface/degradation rate in physiological saline)×100 (2)

In the artificial blood vessel after immobilization of the antithrombin agent, the platelet adhesion rate on the fiber surface can be measured by the following method of measuring the platelet adhesion rate on the fiber surface. The lower the platelet adhesion rate on the fiber surface, the better. The platelet adhesion rate on the fiber surface is preferably less than 20%.

### Method of Measuring Platelet Adhesion Rate on Fiber Surface

The artificial blood vessel is cut along the axial direction and a disk sample with a diameter of 12 mm is prepared by punching using a puncher. The sample piece is placed in a well of a 24-well microplate for cell culture (Sumitomo Bakelite Co., Ltd.) such that the blood-contacting surface faces upward, and a metallic pipe-shaped weight with a wall thickness of 3 mm is loaded thereon. Platelet-rich plasma prepared separately is added to the well such that the number of platelets is about 10⁸ per well. The microplate is left to stand at 37° C. for 2 hours and the sample is then removed therefrom and rinsed with PBS(−) (Nissui), followed by destroying platelets and measuring the activity of generated LDH according to the protocol described for LDH Cytotoxicity Detection kit (Takara Bio Inc.). Based on a calibration curve prepared separately, the number of adherent platelets is determined. As shown in Equation 3, the ratio of the number of platelets after the contact with the sample piece to the number of platelets in the platelet-rich plasma before the contact is determined, to provide the platelet adhesion rate.

Platelet adhesion rate(%)=(number of adherent platelets after the contact/number of platelets in platelet-rich plasma)×100 (3)

### Cellular Adhesiveness

The artificial blood vessel is cut along the axial direction and a disk sample with a diameter of 12 mm is prepared by punching using a puncher. The sample piece is placed in a well of a 24-well microplate for cell culture (Sumitomo Bakelite Co., Ltd.), and a metallic pipe-shaped weight with a wall thickness of 3 mm is loaded thereon. Normal human umbilical vein endothelial cells (Takara Bio Inc.) suspended in DMEM medium supplemented with 10% FCS are added thereto such that 10⁶ cells are contained in the well. The microplate is left to stand at 37° C. for 12 hours, and the sample is then removed therefrom and rinsed with PBS(−) (Nissui), followed by detaching the cells by enzyme treatment and measuring the number of detached cells using an MTT Assay Kit (Funakoshi Corporation). As shown in Equation 4, the ratio of the number of adherent cells to the number of cells plated on the sample is determined, to provide the cell adhesion rate.

Cell adhesion rate(%)=(number of adherent cells/number of cells plated)×100 (4)

### Thrombus Adhesion in Blood Circulation

The artificial blood vessel was cut into a length of 4 cm, and connected to a polyvinyl chloride tube having the same inner diameter as the artificial blood vessel, and a length of 32 cm. Into the tube, 4.5 mL of human fresh blood supplemented with heparin at a final concentration of 0.5 IU/mL was introduced and both ends were immediately sealed to form a loop. The prepared loop was fixed on a frame attached to a rotor operated at a rotation speed of 14 rpm in a thermo-hygrostat drier whose temperature was preliminarily adjusted to 37° C., and rotated for 120 minutes. The loop is then taken out, and the polyvinyl chloride tube is cut to remove blood, followed by rinsing with PBS(−) (Nissui). Thereafter, the presence or absence of thrombi formed in the artificial blood vessel is quantified. The test is carried out with N=3. The same test is carried out using, as a negative control, PBS(−) instead of the human fresh blood. The dry weight of the artificial blood vessel with a length of 4 cm is measured before the test and after the removal of blood and rinsing, and the difference between these measured values is regarded as the thrombus weight, and its mean and standard deviation are calculated. When the mean for the sample is not less than (mean+3×standard deviation) for the negative control, the result is evaluated as “+” and, when the mean for the sample is less than this value, the result is evaluated as “−”. When leakage of blood is found through the artificial blood vessel during the circulation, the result is evaluated as “leakage” irrespective of the amount of blood leaked, and the test is stopped.

### Examples

Examples of the artificial blood vessel are concretely described below in detail.

### Examples

A tubular fabric was prepared as a plain-weave tissue using polyethylene terephthalate of 55 Dtex-48 f as a warp, and polymer array fiber of 245 Dtex-40 f as a weft. The polymer array fiber used therefor was composed of 20 parts of polystyrene as sea-component and 80 parts of polyethylene terephthalate as island-component, and the number of islands was 36/f. This tubular fabric was sufficiently treated with an aqueous sodium hydroxide solution at 80° C., and immersed in toluene. Subsequently, the fabric was subjected to raising by using a raising machine, and then to water-jet punching.

The tubular fabric after the treatments described above was treated with 0.5% aqueous sodium hydroxide solution and then subjected to oxidation treatment with 5% potassium permanganate. Subsequently, the tubular fabric was immersed in 1 to 50 mg/mL solutions of the compounds of General Formulae (V) to (VIII), which were produced by introducing an amino group to the end of the hydrophilic polymer chain contained in General Formulae (I) to (IV). A condensation reaction was allowed to proceed in the presence of 0.1% carbodiimide and the fabric was then rinsed with physiological saline to provide an antithrombotic tubular fabric to be used as an artificial blood vessel.

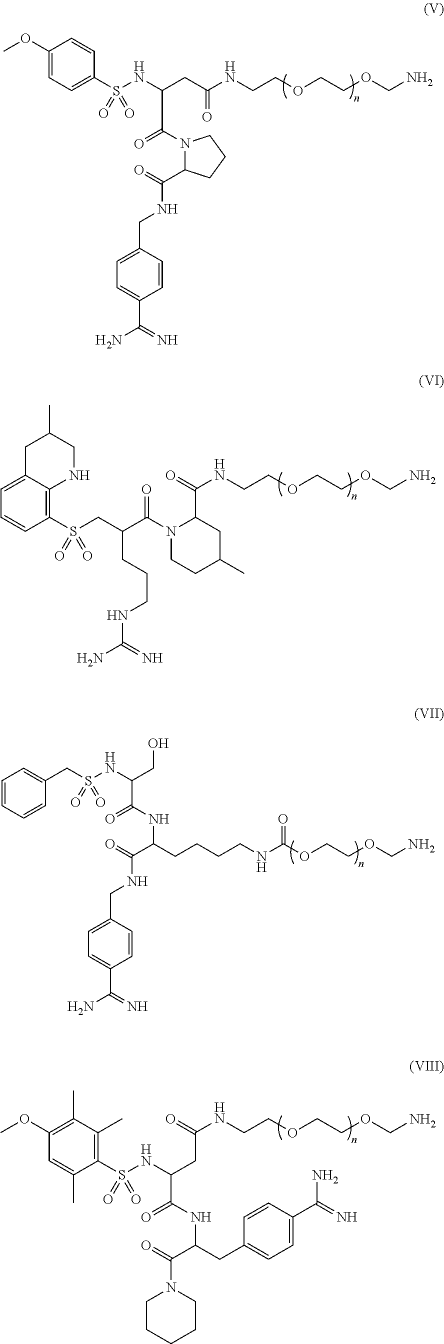

Table 1 shows the performance evaluation results of each antithrombotic tubular fabric obtained by measurement of the water permeability, the thrombin activity inhibition rate in an extract, the thrombin activity inhibition rate on the fiber surface, the platelet adhesion rate, the cell adhesion rate, and thrombus adhesion.

The antithrombotic tubular fabric treated in 1 mg/mL of the antithrombin agent of General Formula (V) was provided as Example 1; the antithrombotic tubular fabric treated in 2 mg/mL of the antithrombin agent of General Formula (V) was provided as Example 2; the antithrombotic tubular fabric treated in 5 mg/mL of the antithrombin agent of General Formula (V) was provided as Example 3; the antithrombotic tubular fabric treated in 10 mg/mL of the antithrombin agent of General Formula (V) was provided as Example 4; the antithrombotic tubular fabric treated in 20 mg/mL of the antithrombin agent of General Formula (V) was provided as Example 5; the antithrombotic tubular fabric treated in 50 mg/mL of the antithrombin agent of General Formula (V) was provided as Example 6; the antithrombotic tubular fabric treated in 50 mg/mL of the antithrombin agent of General Formula (VI) was provided as Example 7; the antithrombotic tubular fabric treated in 50 mg/mL of the antithrombin agent of General Formula (VII) was provided as Example 8; and the antithrombotic tubular fabric treated in 50 mg/mL of the antithrombin agent of General Formula (VIII) was provided as Example 9.

### Comparative Examples

The same tubular fabric as that obtained in Example 1 was immersed in 50 mg/mL aqueous solutions of the antithrombin agents of General Formulae (I) to (IV), and, in this state, irradiated with γ-ray at 5 kGy at Koga Isotope. Each fabric was washed with Triton-X100 and water, to provide an antithrombotic tubular fabric.

The antithrombotic tubular fabric irradiated with γ-ray in 50 mg/mL of the antithrombin agent of General Formula (I) was provided as Comparative Example 1; the antithrombotic tubular fabric irradiated with γ-ray in 50 mg/mL of the antithrombin agent of General Formula (II) was provided as Comparative Example 2; the antithrombotic tubular fabric irradiated with γ-ray in 50 mg/mL of the antithrombin agent of General Formula (III) was provided as Comparative Example 3; and the antithrombotic tubular fabric irradiated with γ-ray in 50 mg/mL of the antithrombin agent of General Formula (IV) was provided as Comparative Example 4. Table 1 shows the performance evaluation results of each antithrombotic tubular fabric obtained by measurement of the water permeability, the thrombin activity inhibition rate in an extract, the thrombin activity inhibition rate on the fiber surface, the platelet adhesion rate, the cell adhesion rate, and thrombus adhesion.

A tubular fabric was prepared as a high-density plain-weave tissue using polyethylene terephthalate of 55 Dtex-48 f as a warp and polymer array fiber of 245 Dtex-40 f as a weft. The polymer array fiber used therefor was composed of 20 parts of polystyrene as sea-component and 80 parts of polyethylene terephthalate as island-component, and the number of islands was 36/f. This tubular fabric was sufficiently treated with hot water containing NaOH at 80° C., and immersed in toluene. Subsequently, the fabric was subjected to raising by using a raising machine, and then to water-jet punching. The fabric was then immersed in 50 mg/mL aqueous solution of the antithrombin agent of General Formula (I), and, in this state, irradiated with γ-ray at 5 kGy at Koga Isotope. The fabric was washed with Triton-X100 and water, to provide an antithrombotic tubular fabric (Comparative Example 5). Table 1 shows the performance evaluation results of the antithrombotic tubular fabric obtained by measurement of the water permeability, the thrombin activity inhibition rate in an extract, the thrombin activity inhibition rate on the fiber surface, the platelet adhesion rate, the cell adhesion rate, and thrombus adhesion.

A tubular fabric was prepared as a low-density plain-weave tissue using polyethylene terephthalate of 55 Dtex-48 f as a warp and polymer array fiber of 245 Dtex-40 f as a weft. The polymer array fiber used therefor was composed of 20 parts of polystyrene as sea-component and 80 parts of polyethylene terephthalate as island-component, and the number of islands was 36/f. This tubular fabric was sufficiently treated with hot water containing NaOH at 80° C., and immersed in toluene. Subsequently, the fabric was subjected to raising by using a raising machine, and then to water jet punching. The fabric was then immersed in 50 mg/mL aqueous solution of the antithrombin agent of General Formula (I), and, in this state, irradiated with γ-ray at 5 kGy at Koga Isotope. The fabric was washed with Triton-X100 and water, to provide an antithrombotic tubular fabric (Comparative Example 6). Table 1 shows the performance evaluation results of the antithrombotic tubular fabric obtained by measurement of the water permeability, the thrombin activity inhibition rate in an extract, the thrombin activity inhibition rate on the fiber surface, the platelet adhesion rate, the cell adhesion rate, and thrombus adhesion.

A tubular fabric was prepared as a plain-weave tissue using polyethylene terephthalate of 55 Dtex-48 f both as a warp and as a weft. The fabric was immersed in 50 mg/mL aqueous solution of the antithrombin agent of General Formula (I) and, in this state, irradiated with γ-ray at 5 kGy at Koga Isotope. The fabric was washed with Triton-X100 and water to provide an antithrombotic tubular fabric (Comparative Example 7). Table 1 shows the performance evaluation results of the antithrombotic tubular fabric obtained by measurement of the water permeability, the thrombin activity inhibition rate in an extract, the thrombin activity inhibition rate on the fiber surface, the platelet adhesion rate, the cell adhesion rate, and thrombus adhesion.

To a solution of the compound of General Formula (IX) in dimethylformamide, 4 N hydrochloric acid/1,4-dioxane (Toyo Kasei Co., Ltd.) was added dropwise to allow the reaction to proceed, to obtain the hydrochloric acid salt of the General Formula (IX). To the solution of the hydrochloric acid salt in dimethylformamide, dicyclohexylcarbodiimide and 4-hydroxybenzotriazole were added, and polyether-modified silicone (X-22-3939A, Shin-Etsu Chemical Co., Ltd.) was further added thereto, followed by allowing the reaction to proceed. The resulting reaction liquid was then placed in a dialysis tube (Spectra/Por RC Por 6, MWCO=1000), and dialyzed against 10 volumes of distilled water. The reaction liquid after the dialysis was filtered, and the solvent in the filtrate was removed, followed by drying the resultant to obtain a hydrophilic polymer compound. The fabric was then immersed in 50 mg/mL aqueous solution of the hydrophilic polymer compound obtained and, in this state, irradiated with γ-ray at 5 kGy at Koga Isotope. The fabric was washed with Triton-X100 and water to provide an antithrombotic tubular fabric (Comparative Example 8). Table 1 shows the performance evaluation results of the antithrombotic tubular fabric obtained by measurement of the water permeability, the thrombin activity inhibition rate in an extract, the thrombin activity inhibition rate on the fiber surface, the platelet adhesion rate, the cell adhesion rate, and thrombus adhesion.

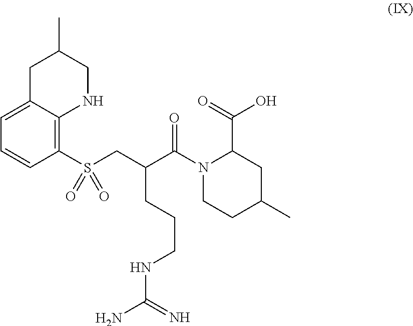 [SMILES: CC1CNc2c(cccc2S(=O)(=O)CC(CCCNC(=N)N)C(=O)N2CCC(C)CC2C(=O)O)C1]

TABLE 1

| | | | | | | | Concentra- | | | | | |
|---|---|---|---|---|---|---|---|---|---|---|---|---|
| | | | | | | | tion of | | Thrombin | | | |
| | | | | | | | antithrombin | Thrombin | activity | | | |
| | | | | | | | activity | activity | inhibition | | | |
| | | | | | | | substance | inhibition | rate | Platelet | Cell | Throm- |
| | | | | Anti- | Hydro- | Water | used for | rate in | on fiber | adhe- | adhe- | bus |
| | | | | thrombin | philic | permeability | treatment | extract | surface | sion | sion | adhe- |
| | | Sample | | agent | polymer | (mL/cm²/min) | (mg/mL) | (%) | (%) | rate (%) | rate (%) | sion |
| Example | 1 | PET fiber | Ultrafine fiber | V | PEG | 130 | 1 | 0.3 | 68 | 4 | 84 | − |
| | 2 | PET fiber | Ultrafine fiber | V | PEG | 1400 | 2 | 0.3 | 73 | 6 | 88 | − |
| | 3 | PET fiber | Ultrafine fiber | V | PEG | 2330 | 5 | 0.2 | 77 | 3 | 87 | − |
| | 4 | PET fiber | Ultrafine fiber | V | PEG | 3420 | 10 | 0.5 | 84 | 8 | 92 | − |
| | 5 | PET fiber | Ultrafine fiber | V | PEG | 2100 | 20 | 0.1 | 89 | 8 | 83 | − |
| | 6 | PET fiber | Ultrafine fiber | V | PEG | 2080 | 50 | 0.2 | 90 | 8 | 91 | − |
| | 7 | PET fiber | Ultrafine fiber | VI | PEG | 2410 | 50 | 0.9 | 93 | 6 | 94 | − |
| | 8 | PET fiber | Ultrafine fiber | VII | PEG | 2250 | 50 | 1.2 | 90 | 1 | 89 | − |
| | 9 | PET fiber | Ultrafine fiber | VIII | PEG | 2160 | 50 | 0.6 | 87 | 4 | 90 | − |
| Compara- | 1 | PET fiber | Ultrafine fiber | I | — | 3390 | 50 | 0.6 | 56 | 14 | 83 | + |
| tive | 2 | PET fiber | Ultrafine fiber | II | — | 2490 | 50 | 0.3 | 41 | 7 | 72 | + |
| Example | 3 | PET fiber | Ultrafine fiber | III | — | 2250 | 50 | 0.4 | 43 | 11 | 86 | + |
| | 4 | PET fiber | Ultrafine fiber | IV | — | 2730 | 50 | 0.3 | 38 | 10 | 81 | + |
| | 5 | PET fiber | Ultrafine fiber | I | — | 50 | 50 | 0.2 | 30 | 16 | 54 | − |
| | 6 | PET fiber | Ultrafine fiber | I | — | 4380 | 50 | 0.2 | 52 | 28 | 34 | Blood |
| | | | | | | | | | | | | leakage |
| | 7 | PET fiber | — | I | — | 4610 | 50 | 0.2 | 35 | 21 | 12 | Blood |
| | | | | | | | | | | | | leakage |
| | 8 | PET fiber | Ultrafine fiber | I | — | 2510 | 50 | 0.2 | 31 | 21 | 75 | − |

As shown in Table 1, when an antithrombin agent was immobilized by condensation reaction, the antithrombin activity on the fiber surface of the artificial blood vessel was high, and no thrombus formation occurred during the circulation. On the other hand, when an antithrombin agent was immobilized by γ-ray irradiation, the surface antithrombin activity was low, and thrombus formation occurred. When the water permeability was high, blood leakage occurred during the circulation. Also, when immobilization was carried out in the absence of a spacer, thrombus formation occurred. When an antithrombin agent is immobilized by condensation reaction, the surface treatment can be achieved also in deep portions of the microstructure of the fiber so that the effect to suppress thrombus formation is high.

### INDUSTRIAL APPLICABILITY

Our artificial blood vessels have both antithrombogenicity and cellular affinity, promote intimal formation after indwelling and maintain antithrombogenicity during intimal formation and can maintain their patency for a long time.

1.-12. (canceled)

13. An artificial blood vessel comprising a tubular fabric comprising a fiber layer containing an ultrafine fiber(s) and an ultrafine fiber layer inside of the fiber layer, the ultrafine fiber layer composed of an ultrafine fiber(s) having a fiber diameter(s) of not less than 10 nm and not more than 3 μm, wherein
an antithrombin agent having a polymer chain other than heparin is covalently bound to said ultrafine fiber via said polymer chain, and
a thrombin activity inhibition rate on the fiber surface at 37° C. is not less than 60%.

14. The artificial blood vessel according to claim 13, wherein the molecular weight of said antithrombin agent is not more than 3000.

15. The artificial blood vessel according to claim 13, whose water permeability at 120 mmHg is not less than 100 mL/cm²/min and less than 4000 mL/cm²/min.

16. The artificial blood vessel according to claim 13, wherein the thrombin activity inhibition rate in an extract obtained by 24 hours of extraction at 37° C. in 10 mL of physiological saline per 1 g of said artificial blood vessel is less than 5%.

17. The artificial blood vessel according to claim 13, wherein said antithrombin agent has one or more of a guanidino group, guanido group, and amidino group.

18. The artificial blood vessel according to claim 13, wherein said polymer chain is a polymer structure selected from polyalkylene glycol, polyvinyl alcohol, and polyvinyl pyrrolidone.

19. The artificial blood vessel according to claim 13, wherein said antithrombin agent is selected from the group consisting of Chemical Formulae (I) to (IV): 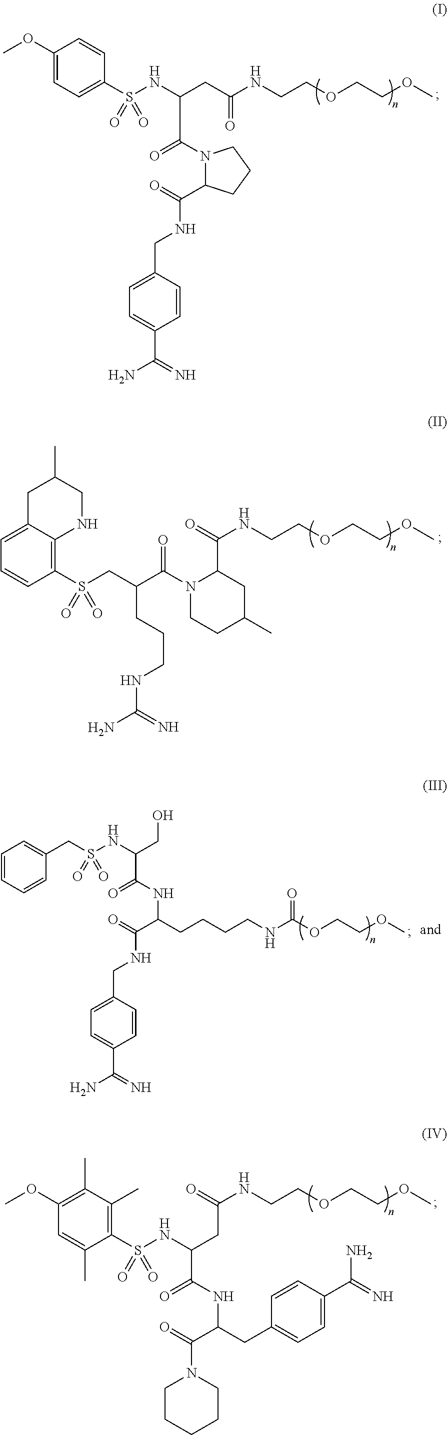
wherein n represents an integer of 1 to 500.

20. The artificial blood vessel according to claim 13, wherein said fiber layer is composed of said ultrafine fiber(s) and a multifilament(s), the multifilament(s) having a total fineness of 1 to 60 decitex.

21. The artificial blood vessel according to claim 20, wherein the fineness of single yarns constituting said multifilament is 0.5 to 10.0 decitex.

22. The artificial blood vessel according to claim 13, having a platelet adhesion rate of less than 20%.

23. The artificial blood vessel according to claim 13, wherein said tubular fabric is composed of a polyester fiber(s).

24. The artificial blood vessel according to claim 13, wherein an inner diameter of said tubular fabric is not less than 1 mm and less than 10 mm.
